(19) United States
(12) Patent Application Publication
(10) Pub. No.: US 2006/0009426 A1
(43) Pub. Date: Jan. 12, 2006

(54) Pharmaceutical compositions comprising calcitriol and a clobetasol propionate

(75) Inventors: Andre Jomard, Saint Vallier De Thiey (FR); Olivier Roye, Mouans Sartoux (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: GALDERMA RESEARCH & DEVELOPMENT, S.N.C., VALBONNE SOPHIA ANTIPOLIS (FR)

(21) Appl. No.: 11/154,706

(22) Filed: Jun. 17, 2005

Related U.S. Application Data
(63) Continuation of application No. PCT/EP03/15011, filed on Dec. 11, 2003.
(60) Provisional application No. 60/437,057, filed on Dec. 31, 2002.

(30) Foreign Application Priority Data
Dec. 17, 2002 (FR) 02/16016

(51) Int. Cl. A61K 31/59; A61K 31/573

(52) U.S. Cl. 514/167; 514/171

(57) ABSTRACT

Topically applicable pharmaceutical gel, cream, lotion, solution or ointment compositions contain synergistically effective amounts of a) calcitriol and b) clobetasol propionate, formulated into a topically applicable, pharmaceutically acceptable medium therefor, and are useful for the treatment of such dermatological afflictions or conditions as psoriasis, atopic dermatitis, contact dermatitis and seborrhoeic dermatitis.

### CROSS-REFERENCE TO PRIORITY/PCT/PROVISIONAL APPLICATIONS

This application claims priority under 35 U.S.C. § 119 of FR 02/16016, filed Dec. 17, 2002, and of provisional application Ser. No. 60/437,057, filed Dec. 31, 2002, and is a continuation of PCT/EP 2003/015011, filed Dec. 11, 2003 and designating the United States (published in the English language on Jul. 1, 2004 as WO 2004/054588 A1), each hereby expressly incorporated by reference and each assigned to the assignee hereof.

### CROSS-REFERENCE TO COMPANION APPLICATIONS

Copending applications Ser. No. 10/942,997, filed Sep. 17, 2004; Ser. No. 10/944,887, filed Sep. 21, 2004; Ser. No. 60/634,105, filed, Dec. 8, 2004; and Ser. No. 11/017,665, filed Dec. 22, 2004.

### BACKGROUND OF THE INVENTION

1. Technical Field of the Invention

The present invention relates to pharmaceutical compositions formulated as a gel, a cream, a lotion, a solution or an ointment comprising, in a pharmaceutically acceptable medium, at least calcitriol and clobetasol propionate, and to the use of those compositions for the preparation of a medicinal product for treating dermatological complaints, afflictions or conditions such as psoriasis, atopic dermatitis, contact dermatitis and seborrhoeic dermatitis.

More specifically, the present invention relates to pharmaceutical compositions comprising at least calcitriol and clobetasol propionate, in a given ratio such that a synergistic effect between the two active principles is observed in the treatment of dermatological complaints, afflictions or conditions such as psoriasis, atopic dermatitis, contact dermatitis and seborrhoeic dermatitis.

2. Description of Background and/or Related and/or Prior Art

The combination of active principles is not administered conventionally in the treatment of dermatological complaints, afflictions or conditions. This is generally due to the difficulty encountered by one skilled in the art during the combination of two active principles as regards the chemical stability and the interactions that the medicinal products may elicit when they are present in the same formulation.

Calcitriol is a vitamin D analogue used to regulate the level of calcium in the body. Its use in the treatment of dermatological diseases has especially been described in U.S. Pat. No. 4,610,978 for the treatment of psoriasis. This said patent suggests compositions comprising calcitriol that may also contain an amount of an anti-inflammatory agent such as clobetasol propionate; however, no specific embodiment for combining calcitriol and a corticosteroid is described or tested in terms of efficacy. Consequently, it would not have been obvious to one skilled in this art to anticipate that the combination of calcitriol with clobetasol propionate would have any synergistic effect.

WO 00/64450 mentions the use of a pharmaceutical composition containing a vitamin D analogue and clobetasol propionate. All the examples of compositions in said patent application combine calcipotriol and betamethasone. The comparison of the measurements of the efficacy, on patients suffering from psoriasis of a composition comprising calcipotriol alone, betamethasone alone or a combination of the two active agents shows that the effect obtained by the combination corresponds to an additive effect. Thus, with regard to this document, one skilled in the art could not in any way have imagined that the combination of a vitamin D analogue with a corticosteroid could have a synergistic effect.

### SUMMARY OF THE INVENTION

It has now surprisingly been determined that the combination of calcitriol with clobetasol propionate affords a synergistic effect in the treatment of certain dermatological complaints, afflictions or conditions such as psoriasis, atopic dermatitis, contact dermatitis and seborrhoeic dermatitis.

Thus, the present invention features pharmaceutical compositions in the form of a gel, a cream, a lotion or a solution or ointment for topical use, comprising, formulated into a pharmaceutically acceptable medium:
a) calcitriol, and
b) clobetasol propionate.

The calcitriol and the clobetasol propionate are present in the compositions according to the invention in an amount such that they act synergistically to impart to the composition a therapeutic effect higher than the theoretical effect obtained by adding together the effects obtained by each of the two active agents taken separately.

### DETAILED DESCRIPTION OF BEST MODE AND SPECIFIC/PREFERRED EMBODIMENTS OF THE INVENTION

More particularly according to the present invention, it has now surprisingly been demonstrated that when thus combined in the same composition, calcitriol and clobetasol propionate are more effective in the treatment of dermatological complaints, afflictions or conditions than if they are administered separately.

The synergistic effect between these two active principles was observed in a model of delayed hypersensitivity reaction in mice, which constitutes an immunological response of Th1 type. This test represents a reliable model for predicting the immunomodulatory properties of a product in a pathology of Th1 type such as psoriasis.

Thus, low doses of clobetasol propionate are sufficient to have an effective action when it is used in combination with calcitriol, for example, the use of 0.01% of clobetasol propionate results in a reduction of inflammation when it is combined with 0.0003% of calcitriol (FIG. 2), whereas clobetasol propionate alone at this concentration shows moderate efficacy (FIGS. 1 and 2).

In the compositions according to the invention defined above, the calcitriol may be used at concentrations of from 0.0001 to 1 mg/g of composition.

In one preferred embodiment of the invention, the compositions are gels, creams, lotions, solutions or ointments and contain clobetasol propionate at concentrations of from 0.01% to 2% by weight relative to the total weight of the composition.

The present invention also features pharmaceutical compositions containing calcitriol, advantageously at concentrations of from 0.001 to 1 mg/g of composition, and 0.01% of clobetasol propionate by weight relative to the total weight of the composition.

Preferably, the composition as previously described is an ointment, a cream, a lotion a solution or a gel, advantageously an ointment.

The compositions according to the present invention are thus formulated either in the form of creams, lotions, solutions or gels, or in the form of ointments by using a suitable vehicle.

Preferably, the creams may be formed from a mixture of mineral oil, or a mixture of beeswax and water that emulsifies instantaneously, to which is added the calcitriol, dissolved in a small amount of oil such as almond oil.

Preferably, the lotions may be prepared by dissolving the calcitriol and the clobetasol propionate in an alcohol of high molecular mass, such as polyethylene glycol.

The ointments may be formulated by mixing together a solution of calcitriol and of clobetasol propionate in an oil such as almond oil, in heated paraffin, followed by allowing the mixture to cool.

The gels may preferably be prepared by dispersing or dissolving the calcitriol and the clobetasol propionate in a suitable ratio, in a gel of carbomer, poloxamer or cellulosic type.

Other ingredients may be added to the topical composition, such as preservatives, for example DL-α-tocopherol, or fragrances, if necessary.

The present invention also features one of the pharmaceutical compositions as defined above, eliciting a synergistic effect between calcitriol and clobetasol propionate in the treatment of dermatological complaints, afflictions or conditions such as psoriasis, atopic dermatitis, contact dermatitis and seborrhoeic dermatitis.

The present invention also features the use of one of the compositions as defined above for the manufacture of a medicinal product for treating dermatological complaints, afflictions or conditions such as psoriasis, atopic dermatitis, contact dermatitis and seborrhoeic dermatitis, advantageously psoriasis.

The invention also features the use of a pharmaceutical composition in the form of an ointment for topical application comprising, in a pharmaceutically acceptable medium:
a) calcitriol, and
b) clobetasol propionate
   for the manufacture of a medicinal product for treating atopic dermatitis, contact dermatitis and seborrhoeic dermatitis.

The compositions of the invention present the following advantages over the prior art, in the case of treatment of skin complaints, afflictions or conditions:
a composition containing a combination of calcitriol and clobetasol propionate will present the advantage of having better efficacy than the use of calcitriol alone,
the use of a combination of clobetasol propionate and calcitriol makes it possible to shorten the treatment period, whether regime or regimen,
a composition containing a mixture of calcitriol and topical clobetasol propionate, such as clobetasol propionate, makes it possible to use a lower dose of calcitriol, and thus to reduce the side effects of calcitriol (irritation and hypercalcaemia) and also the risks associated with the use of corticosteroids, in particular immune deficiency and functional modifications of the HPA axis (hypothalamo-pituitary-adrenal axis),
the use of a combination of calcitriol and clobetasol propionate reduces the side effects of irritation of calcitriol on sensitive skin such as skin suffering from psoriasis, thus increasing the tolerance of the calcitriol treatment.

### BRIEF DESCRIPTION OF THE FIGURES OF DRAWINGS

FIG. 1: Dose-response effect of the topical application of clobetasol propionate (product B) on the intensity of mouse ear oedema.

The results expressed are the average of seven mice (±SD (standard deviation)). The statistical value was determined using Student's t test (NS: non-significant p>0.05; ***p<0.001).

FIG. 2: Synergistic effect produced by the application of a combination of calcitriol (product A) and clobetasol propionate (product B) on mouse ear oedema.

The results expressed are the average of seven mice (±SD (standard deviation)). The statistical value was determined using Student's t test (NS: non-significant p>0.05; ***p<0.001).

FIG. 3: Dose-response effect of the topical application of clobetasol propionate (product B) on the count of auricular ganglionic cells.

FIG. 4: Synergistic effect produced by the application of a combination of calcitriol (product A) and clobetasol propionate (product B) on the count of ganglionic cells.

FIG. 5: Synergistic effect produced by the application of a combination of calcitriol (product A) and clobetasol propionate (product B) on the cutaneous concentration of IFN-γ.

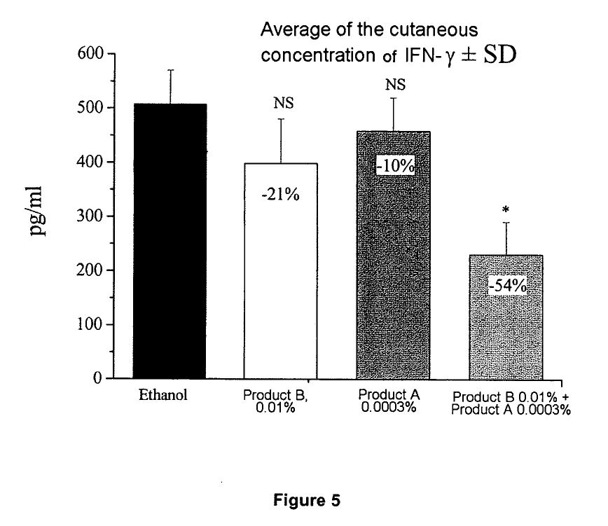

The concentrations are expressed in pg/ml ±SD. The statistical significance was determined using Student's t test (NS: non-significant p>0.05; *p<0.005).

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

### Model of Mice Sensitized with a Hapten

For the purpose of simplicity, in the examples that follow, calcitriol is denoted as product A and clobetasol propionate as product B.

8-week-old Balb/c mice are pretreated on the abdominal skin from day 1 to day 6 using product A or B, or A and B diluted in ethanol. On day 6, the mice are actively sensitized by the topical application of 50 mg of oxazolone (oxa) in ethanol onto the abdominal skin. On day 11, an application of 20 mg of oxa in ethanol is performed on the right ear.

Student's t test was used for the statistical analysis of the results.

### 1.A. Ear Oedema

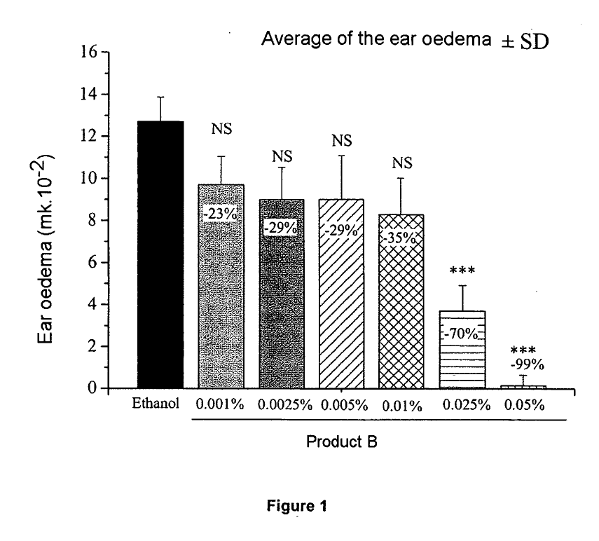

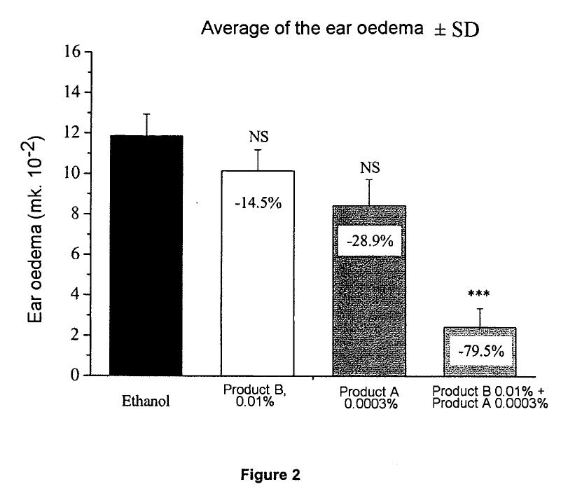

The thickness of the ear is measured, using a micrometer, on day 11 (just before the application of oxazolone to the presensitized mice) and after 24 hours, on day 12. The ear oedema is expressed as the difference between the measurement of the thickness of the ear between day 12 and day 11. The values of the thickness of the ear are analyzed statistically using Student's t test. The experimental results show a dose-response effect for product (FIG. 1). The 0.01% dose was chosen to perform the treatment using the combination of product A and product B, in order to be able to observe a synergistic effect. FIG. 2 shows a synergistic effect during the use of a combination of product A with product B.

### 1.B. Count of the Ganglionic Cells

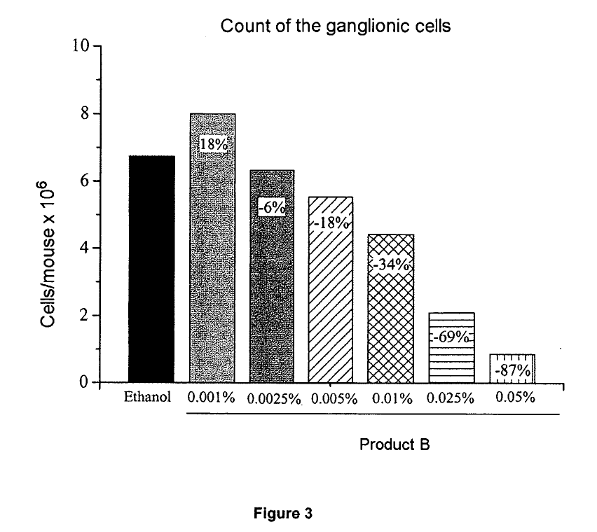

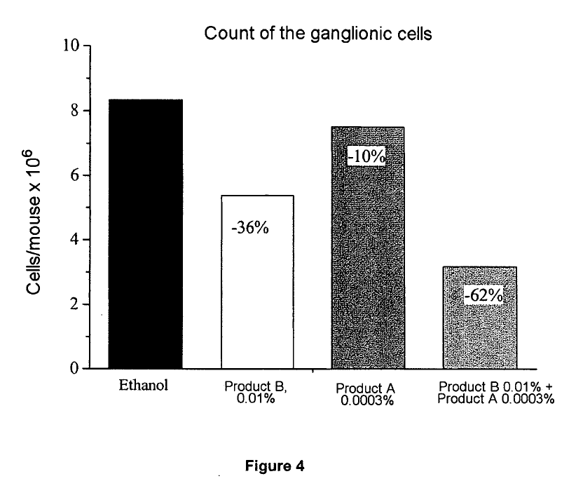

On day 12, one day after the repeated application of the oxa, the animals were sacrificed by cervical dislocation. The ganglionic cells are collected and combined by experimental group. A cell suspension is prepared and the cells are then counted. The number of cells is expressed per animal. FIG. 3 shows a dose-response effect for product B. The 0.01% dose was chosen to perform the treatment using the combination of product A and product B, in order to be able to observe a synergistic effect. FIG. 4 shows a synergistic effect during the use of a combination of product A with product B.

### 1.C. Determination of the Cutaneous Concentration of Interferon-γ

An 8 mm biopsy was performed on day 12 on the ear which received the oxazolone. After homogenization, the IFN-γ content was measured via a standard ELISA test. The results are expressed in pg/ml of homogenizate and the statistical analysis of the results is performed using Student's t test. Interferon-γ is a cytokine of Th1 type, which is strongly expressed in this animal model. The results of the experiments show a synergistic effect during the combination of product A and of product B.

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A topically applicable pharmaceutical gel, cream, lotion, solution or ointment composition, comprising synergistically effective amounts of a) calcitriol and b) clobetasol propionate, formulated into a topically applicable, pharmaceutically acceptable medium therefor.

2. The pharmaceutical composition as defined by claim 1, the calcitriol comprising from 0.001 to 1 mg/g thereof.

3. The pharmaceutical composition as defined by claim 1, the clobetasol propionate comprising from 0.01% to 2% by weight thereof.

4. The pharmaceutical composition as defined by claim 1, formulated as a gel.

5. The pharmaceutical composition as defined by claim 1, formulated as a cream.

6. The pharmaceutical composition as defined by claim 1, formulated as a lotion.

7. The pharmaceutical composition as defined by claim 1, formulated as an ointment.

8. The pharmaceutical composition as defined by claim 1, formulated as a solution.

9. A topically applicable pharmaceutical composition comprising a) calcitriol and about 0.01% by weight of b) clobetasol propionate, formulated into a topically applicable, pharmaceutically acceptable medium therefor.

10. A regime or regimen for treating a dermatological complaint, affliction or condition, comprising topically applying onto the affected skin area of an individual in need of such treatment, a thus effective amount of a topically applicable pharmaceutical gel, cream, lotion, solution or ointment composition which comprises synergistically effective amounts of a) calcitriol and b) clobetasol propionate, formulated into a topically applicable, pharmaceutically acceptable medium therefor.

11. A regime or regimen for treating psoriasis, comprising topically applying onto the affected skin area of an individual in need of such treatment, a thus effective amount of a topically applicable pharmaceutical gel, cream, lotion, solution or ointment composition which comprises synergistically effective amounts of a) calcitriol and b) clobetasol propionate, formulated into a topically applicable, pharmaceutically acceptable medium therefor.

12. A regime or regimen for treating atopic dermatitis, contact dermatitis or seborrhoeic dermatitis, comprising topically applying onto the affected skin area of an individual in need of such treatment, a thus effective amount of a topically applicable pharmaceutical gel, cream, lotion, solution or ointment composition which comprises synergistically effective amounts of a) calcitriol and b) clobetasol propionate, formulated into a topically applicable, pharmaceutically acceptable medium therefor.
